SEQUENCE LISTING (7 sequences)

SEQ ID NO: 1 - protein, 6 aa, Artificial Sequence
  Synthetic Peptide
GAPGPP

SEQ ID NO: 2 - protein, 6 aa, Artificial Sequence
  Synthetic Peptide
GPAGPP

SEQ ID NO: 3 - protein, 6 aa, Artificial Sequence
  Synthetic Peptide
GAPGPA

SEQ ID NO: 4 - protein, 6 aa, Artificial Sequence
  Synthetic Peptide
GAAGPP

SEQ ID NO: 5 - protein, 4 aa, Homo sapiens
VPGG

SEQ ID NO: 6 - protein, 5 aa, Homo sapiens
VPGVG

SEQ ID NO: 7 - protein, 6 aa, Homo sapiens
APGVGV

(19) United States
(12) Patent Application Publication
(10) Pub. No.: US 2007/0054298 A1
(43) Pub. Date: Mar. 8, 2007

(54) Methods for enzyme-mediated coupling of oligomers

(76) Inventors: Kent Kirshenbaum, New York, NY (US); Barney Yoo, New York, NY (US)

Correspondence Address: KLAUBER & JACKSON, 411 HACKENSACK AVENUE, HACKENSACK, NJ, 07601, US

(21) Appl. No.: 11/504,533

(22) Filed: Aug. 14, 2006

Related U.S. Application Data
(60) Provisional application No. 60/707,877, filed on Aug. 12, 2005.
(60) Provisional application No. 60/718,663, filed on Sep. 20, 2005.

(51) Int. Cl. C12Q 1/68; C12P 19/34; C12P 21/06; C12P 17/00

(52) U.S. Cl. 435/6; 435/91.2; 435/69.1; 435/117

(57) ABSTRACT

The present invention is directed to a method for synthesizing sequence specific short and long chain length heteropolymers. More specifically, the method is directed to enzyme-mediated ligation of telechelic sequence specific oligomers to generate abiotic linked oligomers and concatenated synthetic oligomers. The invention also encompasses abiotic linked oligomers and abiotic concatenated oligomers comprising greater than 65 monomer units.

### CROSS REFERENCE TO RELATED APPLICATION

This application claims priority under 35 USC §119(e) from U.S. Provisional Application Ser. No. 60/707,877, filed Aug. 12, 2005, and U.S. Provisional Application Ser. No. 60/718,663, filed Sep. 20, 2005 which applications are herein specifically incorporated by reference in their entirety.

### FIELD OF INVENTION

The present invention relates to novel methods for the synthesis of linked or concatenated abiotic and/or biotic sequence specific oligomers comprising a plurality of repeated oligomer blocks or subunits. More specifically, the present invention is directed to novel methods for synthesizing linked abiotic oligomers comprising homopolymers of the identical oligomers or heteropolymers of different oligomers, or biotic concatenated oligomers, wherein subunit coupling is achieved by enzymatic catalysis.

### BACKGROUND OF INVENTION

Several publications and patent documents are referenced in this application in order to more fully describe the state of the art to which this invention pertains. The disclosure of each of these publications and documents is incorporated by reference herein.

Current coupling strategies for the synthesis of both short and long oligomers generally entail synthesis from small monomer units either through solid or solution phase chemistry. The limitations associated with these methods are either restricted chain lengths or minimal sequence specificity and diversity. Also, current block or segment ligation strategies typically utilize highly reactive, nonspecific, chemical agents or functionalities requiring controlled chemical environments and/or protecting groups—for chemically reactive functionalities on the segment units—during ligation (Tu et al. Advanced Drug Delivery Reviews. 2004, 56, 1537; Deming et al. Nature. 1997, 390, 386; Ayers et al. Macromolecules. 2003, 36, 5967).

Polypeptides, which are natural oligomers, while capable of exhibiting an extraordinary range of bioactivities, often display poor pharmacological properties. For this reason, synthetic mimics of peptides have been the focus of vigorous development by medicinal and bioorganic chemists. A variety of oligomeric peptidomimetics have been introduced that show potential as partial mimics of natural polypeptide species in that they exhibit some of the structural and functional attributes of natural polypeptides (Patch et al., Curr. Opin. Chem. Biol., 2002, 6, 872). Further elaboration of peptidomimetic structures may lead to a greater range of capabilities for these compounds.

N-substituted glycine oligomers, or peptoids, are an example of a promising class of peptidomimetics. Peptoids are polymers based on a polyamide backbone, which can be produced by an efficient, automated solid-phase synthesis that facilitates the incorporation of diverse N-pendant sidechains in a sequence-specific manner. As such, peptoids are a class of non-natural, sequence-specific polymers that represent an alternative derivative of a polyamide backbone, the sequence and length of which can be precisely controlled. Structurally, peptoids differ from polypeptides in that their sidechains are pendant groups of the amide nitrogen rather than the α-carbon (Simon et al., Proc. Natl. Acad. Sci. USA, 1992, 89, 9367; Zuckermann et al., J. Am. Chem. Soc., 1992, 114, 10646). Peptoids are particularly useful for biomedical applications because these molecules are basically invulnerable to protease degradation and hence are more stable than polypeptides in vivo and less likely to be rendered immunogenic. These properties enhance bioavailability. Moreover, peptoids, which are synthetically produced by definition, can be produced essentially in the absence of impurities.

### SUMMARY OF INVENTION

The invention provides a general method for enzyme catalyzed ligation of oligomers. This approach employs the incorporation of an enzyme recognition element into a ‘block’ oligomer(s), followed by enzyme-mediated ligation of two or more of the block oligomers. More particularly, the method is directed to enzyme-mediated ligation of two or more telechelic oligomers. As understood in the art, a telechelic oligomer is a prepolymer capable of entering into further polymerization via its reactive end-groups (Definition 1.11 in IUPAC. Glossary of Basic Terms in Polymer Science, Pure Appl. Chem., 68, 2287-2311 (1996)). The resulting polymer product or linked oligomer is a concatemer of varying chain lengths ranging in size from 2- to n-oligomer blocks. See FIGS. 1A and 1B.

The present invention is directed to a method for linking telechelic sequence-specific oligomers, the method comprising: providing a first abiotic telechelic oligomer comprising a first enzyme recognition element; providing a second telechelic oligomer; contacting the first abiotic telechelic oligomer and second telechelic oligomer with an enzyme capable of recognizing the first enzyme recognition element under mild conditions effective to link the first abiotic telechelic oligomer and second telechelic oligomer proximate to the enzyme recognition element of the first abiotic telechelic oligomer, thereby forming an abiotic linked oligomer comprising the first abiotic telechelic oligomer and second telechelic oligomer. As described herein, the first abiotic telechelic oligomer and the second telechelic oligomer are ligated in a head to tail fashion. More specifically, the carboxy terminus of the first abiotic telechelic oligomer is ligated to the amino terminus of the second telechelic oligomer.

In an embodiment of the present method, the first abiotic telechelic oligomer is selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas. [Cheng, Current Opinion In Structural Biology, 2004, 14 (4): 512-520].

In an aspect of the present method, the second telechelic oligomer is an abiotic telechelic oligomer. In a further embodiment of the present method, the first and second abiotic telechelic oligomers are selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas. The first and second abiotic telechelic oligomers may comprise different monomeric sequences or may comprise identical monomeric sequences. As described herein, the first and second abiotic telechelic oligomers are ligated in a head to tail fashion.

In an aspect of the present method, the enzyme that recognized the enzyme recognition element is a protease, lipase, esterase, glycosidase, or transaminase. In a particular aspect of the method, the enzyme is a protease and the enzyme recognition element is a compatible protease recognition element. In a further aspect of the method, the protease is selected from the group consisting of clostripain, trypsin, chymotrypsin, V8 protease, subtilisin, subtiligase, collagenase, thermolysin, papain, SGBP, and dipeptidyl peptidase.

In another embodiment of the method, the second oligomer comprises a second enzyme recognition element. In an aspect of the method, the first enzyme recognition element and the second enzyme recognition element are identical. The method of the invention may comprise repeating the contacting/linking step one or more times to generate an abiotic linked oligomer comprising at least 3 telechelic oligomers. Such methods are useful for generating abiotic linked oligomers comprising 3 to 500 telechelic oligomers.

In another aspect of the method, the second abiotic telechelic oligomer comprises a second enzyme recognition element. In a particular embodiment of the method, the first enzyme recognition element and the second enzyme recognition element are identical. In a more particular embodiment, the first and second enzyme recognition elements are protease enzyme recognition elements and the enzyme used is a protease. The protease that recognizes the first and second enzyme recognition elements may be selected from the group consisting of clostripain, trypsin, chymotrypsin, V8 protease, subtilisin, subtiligase, collagenase, thermolysin, papain, SGBP, and dipeptidyl peptidase.

In an aspect of the present invention, the method comprises repeating the contacting/linking step one or more times to generate an abiotic linked oligomer comprising at least 3 telechelic abiotic oligomers. Such methods are useful for generating abiotic linked oligomers comprising 3 to 500 telechelic abiotic oligomers. The abiotic linked oligomer may comprise first and second abiotic telechelic oligomers, wherein the first and second abiotic telechelic oligomers comprise different monomeric sequences or comprise identical monomeric sequences.

The present invention also encompasses an abiotic linked oligomer comprising: a plurality of first abiotic telechelic oligomers and a plurality of second telechelic oligomers, wherein the first abiotic telechelic oligomers and second telechelic oligomers are linked and wherein the abiotic linked oligomer is greater than 65 monomers long. Such abiotic linked oligomers of the invention may comprise 2 to 500 first abiotic telechelic oligomers and second telechelic oligomers.

The first abiotic telechelic oligomer of such an abiotic linked oligomer is selected from the group consisting of N-substituted glycine peptoid oligomers, beta-peptoids, beta-peptides, gamma-peptides, sulfonamidopeptides, N-alkylated peptides, polyesters, peptide nucleic acids and oligoureas. In a particular embodiment, the second telechelic oligomers are also abiotic telechelic oligomers. In a more particular embodiment, the first and second abiotic telechelic oligomers are selected from the group consisting of N-substituted glycine peptoid oligomers, beta-peptoids, beta-peptides, gamma-peptides, sulfonamidopeptides, N-alkylated peptides, polyesters, peptide nucleic acids and oligoureas.

The present invention encompasses embodiments wherein the abiotic linked oligomer comprises first and second abiotic telechelic oligomers and the first and second abiotic telechelic oligomers comprise different monomeric sequences or identical monomeric sequences.

In an embodiment wherein the first and second telechelic oligomers are abiotic telechelic oligomers, the abiotic linked oligomer may comprise 2 to 500 abiotic telechelic oligomers.

In a further aspect, the invention is directed to a method for concatenating telechelic sequence specific oligomers, the method comprising: providing at least one first telechelic sequence specific oligomer comprising a first enzyme recognition element; providing at least one second telechelic sequence specific oligomer comprising the first enzyme recognition element; contacting the at least one first telechelic sequence specific oligomer and the at least one second telechelic sequence specific oligomer with an enzyme capable of recognizing the first enzyme recognition element under mild conditions effective to link first telechelic sequence specific oligomers and second telechelic sequence specific oligomers proximate to the enzyme recognition element of either the first telechelic sequence specific oligomer or the second telechelic sequence specific oligomer, thereby forming a concatenated oligomer comprising at least 3 first and second telechelic sequence specific oligomers.

In an embodiment, the first and second telechelic sequence specific oligomers comprise identical monomeric sequences. In a different embodiment, the first and second telechelic sequence specific oligomers comprise different monomeric sequences. Moreover, it is to be understood that the ratio of the first and second telechelic sequence specific oligomers which comprise different monomeric sequences may be varied according to the desired product (i.e., concatenated oligomer).

In accordance with the invention, the enzyme used to concatenate telechelic sequence specific oligomers is an oxidoreductase, a peroxidase, a laccase, a transferase, a glycotransferase, a phosphorylase, a glycosyl transferase, an acyltransferase, a hydrolase, a ligase, a protease, a lipase, an esterase, a glycosidase, and a transaminase. Engineered and mutated versions of any of these natural enzymes are also envisioned in the present method.

In a particular embodiment, the enzyme used to concatenate telechelic sequence specific oligomers is a protease and the first enzyme recognition element is a protease recognition element recognized by the protease. Exemplary proteases envisioned for use in the present include clostripain, trypsin, chymotrypsin, V8 protease, subtilisin, subtiligase, collagenase, thermolysin, papain, SGBP, and dipeptidyl peptidase.

In a further aspect of the invention, the method further comprises contacting the concatenated oligomer comprising at least 3 first and second telechelic sequence specific oligomers with at least one third telechelic sequence specific oligomer comprising the first enzyme recognition element, said contacting the concatenated oligomer and the at least one third telechelic sequence specific oligomer with an enzyme capable of recognizing the first enzyme recognition element under mild conditions effective to link the concatenated oligomer and the at least one third telechelic sequence specific oligomer proximate to the enzyme recognition element of either of the concatenated oligomer or the at least one third telechelic sequence specific oligomer, thereby forming a concatenated oligomer comprising at least 3 first and second telechelic sequence specific oligomers and at least one third telechelic sequence specific oligomer. The third telechelic sequence specific oligomer may comprise a monomeric sequence that is identical to or different from that of the first or second telechelic sequence specific oligomer. It is to be understood that additional cycles of concatenation may be performed to ligate additional telechelic sequence specific oligomers to previously synthesized concatenated oligomers.

### BRIEF DESCRIPTION OF THE DRAWINGS

FIGS. 1A and 1B show a general scheme of the invention. Oligomer X can be an oligomer of any size and sequence, either different from or identical to that of Oligomer A. FIG. 1A depicts a single ligation reaction between two oligomers. FIG. 1B depicts multiple (N) ligation reactions between two oligomers.

FIG. 2 shows a reaction scheme for the concatemerization of peptidomimetic oligomers. (Inset) Chemical structure of the block unit peptidomimetic oligomer.

FIG. 3 shows Scheme 1, wherein reaction conditions are as follows: 0.2 M HEPES (pH 8.0), 100 mM NaCl, 1 mM CaCl₂, 12% DMF, 5 mM 1, 10 mM 2, 1.6 μM clostripain, room temperature (rt), 4 h.

FIG. 4 shows a peptoid ligation reaction monitored by reversed-phase HPLC. 1, 2, and 3 correspond to Scheme 1 entries.

FIG. 5 shows Scheme 2, wherein reaction conditions are as follows: 0.2 M HEPES (pH 8.0), 100 mM NaCl, 1 mM CaCl₂, 12% DMF, 5 mM 1, 10 mM 4, 1.6 μM clostripain, rt, 8 h.

FIG. 6 shows Scheme 3, wherein reaction conditions are as follows: 0.2 M HEPES (pH 8.0), 100 mM NaCl, 1 mM CaCl₂, 12% DMF, 5 mM 6, 1.6 μM clostripain, rt, 12 h; n=1,2,3, . . . >30.

FIGS. 7A and B show peptoid concatemer reaction. FIG. 7A shows HPLC of peptoid concatemer reaction. FIG. 7B shows MALDI-mass spectrometry of peptoid concatemer products 7. Average Δm/z=581, corresponding to mass of oligomer repeat units.

FIG. 8 shows a reaction scheme for the generation of peptoid concatemers containing an azide chemical group.

FIGS. 9A and 9B show graphs depicting HPLC analyses of the concatemer reaction as described in FIG. 8. FIG. 9A is a graph depicting the reaction after 10 minutes, whereas FIG. 9B is a graph depicting the reaction after 12 hours.

FIG. 10 shows a reaction scheme for the generation of elastin mimetic peptide concatemers.

FIGS. 11A and 11B show graphs depicting HPLC analyses of the concatemer reaction as described in FIG. 10. FIG. 11A depicts the reaction at the 0 minute time point; FIG. 11B depicts the reaction after 7 hours.

FIG. 12 shows a reaction scheme for the ligation of a peptide fragment to an abiotic oligomer.

FIGS. 13A and 13B show graphs depicting HPLC analyses of the ligation reaction as described in FIG. 12. FIG. 13A depicts the reaction at the 0 minute time point; FIG. 13B depicts the reaction after 12 hours.

FIG. 14 shows schematics depicting the complexity of substrate recognition by an enzyme.

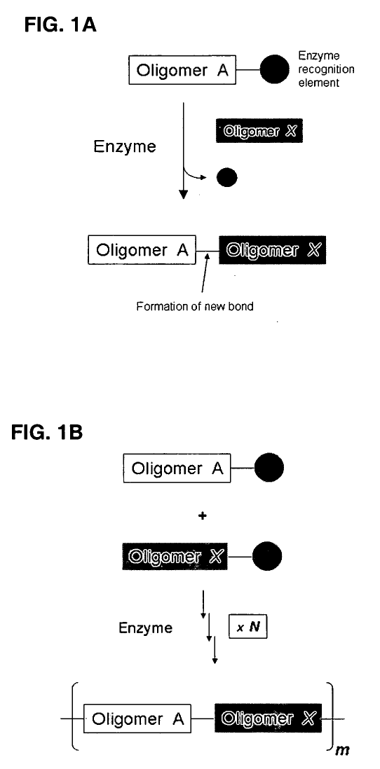

### DETAILED DESCRIPTION OF THE INVENTION

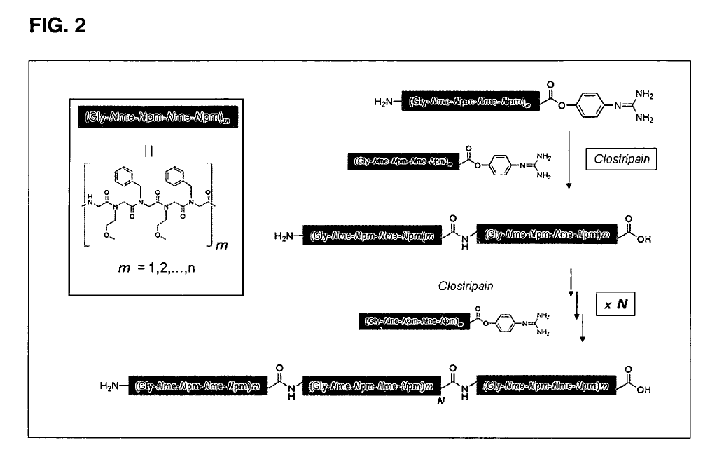

The invention allows for the synthesis of short and long chain length polymers while retaining precise control over sequence specificity in the oligomers. Single telechelic oligomer blocks can be used to generate homopolymers of the telechelic oligomer. Alternatively, different telechelic oligomer blocks can be ligated to form heteropolymers of the various telechelic oligomers. The conditions for synthesis are generally mild, and can be performed in aqueous and aqueous/organic solvents. Enzyme catalysis allows for highly specific bond formation between block units, thus precluding the need for protecting groups during ligation. An application of this invention is described in FIG. 2. As shown in FIG. 2, a concatemer of a sequence specific peptidomimetic oligomer (pentamer) was synthesized. In this embodiment, the enzyme used for ligation of peptidomimetic oligomer is Clostripain, a cysteine protease specific for basic amino acid residues (primarily arginine). The peptidomimetic oligomer is functionalized with an enzyme recognition element, para-oxy-guanidino-phenol. The enzyme recognizes the guanidino functionality of the functionalized first oligomer, and subsequently catalyzes amide bond formation between the carboxyl group of the first oligomer with the N-terminal amine of a second oligomer block or unit. Numerous cycles of this ligation event produce a family of concatemers, as shown in the high performance liquid chromatography (HPLC) and matrix assisted laser desorption and ionization time of flight (MALDI-TOF) data shown in FIGS. 7A and 7B. Specifically, MALDI-TOF data confirms the presence of products ranging from two oligomer units to large concatemers of oligomers having masses greater than 19 kDa.

Accordingly, the invention enables synthesis of large natural and unnatural polymers and biomaterials. One notable application for this methodology is the synthesis of protein materials such as collagen and elastin, along with mimetics of these important biopolymers. Collagen has a wide range of medical applications, including hemostatic materials, biocompatible coatings, drug delivery, and tissue engineering. The present method may be used to incorporate biological and chemical functionalities into a naturally occurring polymer sequence, such as collagen for example, to achieve desired biochemical and mechanical properties. For example, amino acid sequences with biological activity can be utilized; chemical functionalities can be incorporated, followed by subsequent modification—such as covalent attachment of a desired drug compound. Also, introducing specific sequences into natural or unnatural polymers can enable control over the material properties of the polymers. The degree of covalent crosslinking, for example, both intra- and inter-chain, may allow for alterations in elasticity; sequences containing specific chemical functionalities can control the hydrating properties of the polymer material. These potential applications do not exclude chimeric polymers of natural and unnatural sequences. For example, the incorporation of peptidomimetic sequences into the context of normal collagen or elastin polymers. In addition, the mixing of natural and unnatural polymers to form large macromolecular matrices resulting in materials with ‘tunable’ properties is another application with potential therapeutic value.

The methods of the invention differ substantially from those of the prior art in several respects. Firstly, the present method allows for the ligation and polymerization of telechelic sequence-specific oligomers using a protein catalyst or enzyme. As such, the invention does not require the presence of halogenated or metal initiators or sulfhydryl side groups for ligation. Also, the present method does not require a preponderance of organic solvent. For the sake of clarity, the method of the present invention does not encompass the ligation of polypeptides of L- or D-amino acids or other monomers that might be present in natural biological polymers, wherein such ligation produces a naturally occurring oligomer, but is instead directed to the ligation of oligomers, wherein at least one of the linked oligomers is an abiotic oligomer, and to the concatemerization of sequence-specific synthetic oligomers. Secondly, the products of the present method (i.e., linked abiotic oligomers) are macromolecular sequence-specific heteropolymers.

Commonly used approaches for the synthesis of long chain polymers, such as atomic transfer radical polymerization (ATRP), require the use of halogenated initiators and ligand catalysts complexed with transition state metals, all in organic media. Polymers synthesized using α-amino acid-N-carboxyanhydrides (NCA) require only metal initiators. However, these aforementioned approaches provide no control over sequence. Fragment condensation approaches have been used to generate long chain polymers of short oligomer blocks or fragments using standard coupling agents, such as uronium salts and carbodiimides. These reactions are, however, performed in organic media and require the use of protecting groups on chemically sensitive functional groups typically present on side chain positions on the fragment. More recently, the use of native chemical ligation has been applied towards the polymerization of peptide fragments containing pendant sulfhydryl groups at both termini of the fragments.

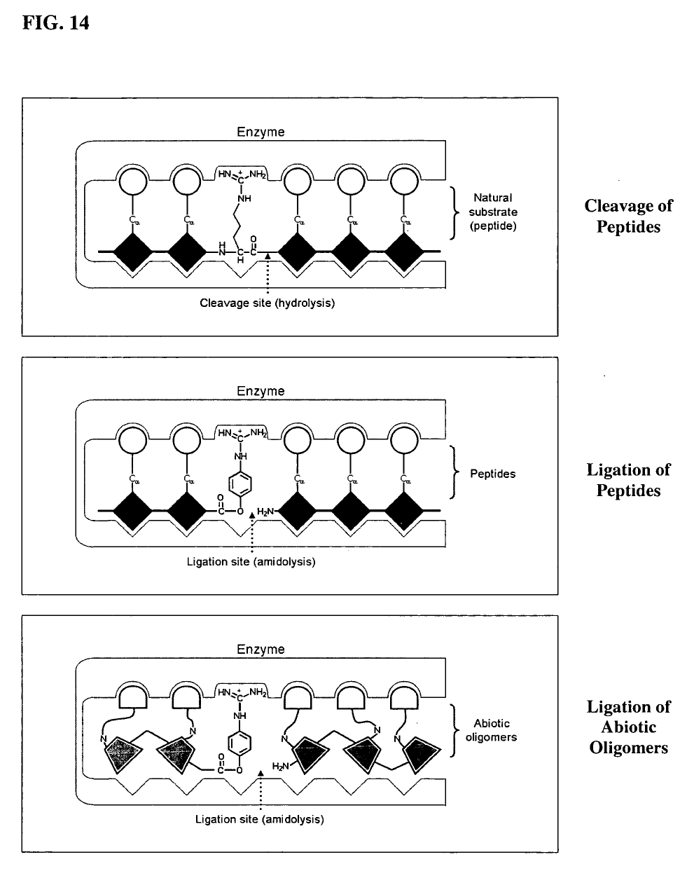

Proteolytic enzymes, or proteases, have been shown to bind to their polypeptide substrates with high specificity and affinity. Research elucidating the function of these enzymes underscores the critical nature of the substrate side chain groups as well as the conformational and chemical features of the substrate backbone. Biochemical studies have shown that the peptide substrate becomes oriented to place multiple side chain groups in registry with specific recognition pockets on the protease surface. This interaction is augmented by formation of hydrogen bond networks between the substrate backbone and the protease. See FIG. 14 for schematic representation of multiple interactions involved in enzyme-substrate recognition.

The present invention reveals and exploits the potential for natural enzymes to catalyze reactions on non-natural oligomeric substrates, despite the absence of characteristic features of polypeptides in these synthetic constructs. The model abiotic oligomer substrates described herein, peptoids, comprise monomers with physico-chemical properties distinct from those of their natural amino acid counterparts. The rearrangement of the pendant groups results in conformational, structural, and chemical properties that are fundamentally distinct from polypeptides. The present invention reveals the unexpected and novel finding that proteases can form productive binding interactions with two abiotic peptoid oligomer fragments, thereby catalyzing their ligation. See FIG. 14 for schematic representation. The present inventors describe conditions under which these reactions occur with substantial efficiency, despite the aforementioned disparities recognized with respect to properties of peptides and peptoids.

In view of the above, the method of the invention enables the synthesis of mimics of large natural and unnatural polymers and biomaterials. One notable application for this methodology is the synthesis of protein materials such as collagen and elastin, along with mimetics of these important biopolymers. Collagen has a wide range of medical applications, from hemostatic materials and biocompatible coatings to drug delivery and tissue engineering. Moreover, the present method may be used to incorporate biological and chemical functionalities into naturally occurring polymer sequences for desired biochemical and mechanical properties. The present method may be used, for example, in conjunction with amino acid sequences with biological activity or chemical functionalities, which may be modified via, for example, covalent attachment of a desired drug compound. Also, introducing specific sequences into natural or unnatural polymers can enable control over the material properties of the polymers. For example, the degree of covalent crosslinking, both intra- and inter-chain crosslinking, may allow for alterations in elasticity; sequences containing specific chemical functionalities can control the hydrating properties of the polymer material. These potential applications do not exclude chimeric polymers of natural and unnatural sequences. For example, the incorporation of peptidomimetic sequences into the context of normal collagen or elastin polymers. In addition, the mixing of natural and unnatural polymers to form large macromolecular matrices resulting in materials with ‘tunable’ properties is another application with potential therapeutic value.

Definitions

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

The terms “peptoid”, “polypeptoid”, “poly(N-substituted glycines)”, “oligo (N-substituted) glycines”, and “oligomeric N-substituted glycines” are used interchangeably herein and are produced using the methodology of the present invention. As indicated herein, peptoids are not peptides in that they are not composed of naturally-occurring amino acids linked by peptide bonds. Peptoids may, however, be designed to possess features (e.g., reactive sites) that structurally mimic naturally occurring peptides and proteins, and as such are useful as potential therapeutic agents and/or as detection reagents. The term “peptoid” or “polypeptoid” refers to a plurality of oligomeric N-substituted glycines of any length. More specifically, a peptoid of the invention is between 2-100 monomers, more particularly between 2-50 or 2-20.

Peptoids can be synthesized in a sequence-specific fashion using an automated solid-phase protocol, e.g., the sub-monomer synthetic route. See, for example, Wallace et al., Adv. Amino Acid Mimetics Peptidomimetics, 1999, 2, 1-51 and references cited therein, all of which are incorporated herein in their entirety by this reference. The synthesis of long peptoids can be achieved using the sub-monomer protocol and such peptoids may comprise alternating methoxy and benzyl side chains.

As indicated above, peptoids (N-substituted glycines) are non-natural, sequence specific polymers composed of a poly-glycine backbone. Peptoid molecules contain functional groups (R) positioned as substituents of the amide nitrogen. A linear peptoid oligomer is distinct from that of a branched peptoid oligomer with respect to the nature of the characteristic “R” group. Linear peptoids contain R groups that do not include peptoid oligomers themselves.

As used herein, the term “oligomer”, “oligomer block”, or “block oligomer” is used to refer to a unit comprising a linear chain of two or more linked monomers. Exemplary abiotic oligomer blocks include N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas. Exemplary monomers include, for example, individual amino acid residues (monomeric units of polypeptides), N-substituted glycines (monomeric units of peptoids), N-substituted beta-alanine (monomeric units of beta-peptoids), aminoxy acids, and monomers comprising the aforementioned exemplary oligomers. The term “oligomer”, “oligomer block”, or “block oligomer” refers to a plurality of monomers of any length. More specifically, an “oligomer”, “oligomer block”, or “block oligomer” of the invention is between 2-100 monomers, more particularly between 2-50 or 2-20.

As used herein, the term “telechelic oligomer” refers to a prepolymer capable of entering into further polymerization via its reactive end-groups (Definition 1.11 in IUPAC. Glossary of Basic Terms in Polymer Science, Pure Appl. Chem., 68, 2287-2311 (1996)). In other words, a telechelic oligomer is an oligomer block, wherein the chemical functionalities at the termini permit the polymerization of the oligomer blocks in a head to tail fashion, including instances where the reactive termini are present but blocked to prevent the polymerization.

As used herein, the term “prepolymer” refers to a polymer or oligomer capable of entering, via reactive groups, into further polymerization; the reactive species thereby contributes more than one monomeric unit to at least one chain of the final polymer (Definition 1.11 in IUPAC. Glossary of Basic Terms in Polymer Science, Pure Appl. Chem., 68, 2287-2311 (1996)).

As used herein, the term abiotic refers to an unnatural or non-natural monomer and/or oligomer unit. This includes chemical fragments that are not present in natural biological systems or living organisms. Abiotic oligomers include monomer units that would not be present in biopolymers synthesized in natural living systems, such as nucleotides present in natural nucleic acids; carbohydrates present in natural polysaccharides; or L- and D-alpha amino acids present in natural polypeptides.

As used herein, the term abiotic linked oligomer refers to a molecule that is generated by ligating or chemically linking oligomer blocks, wherein the ligation or linking is mediated by an enzyme (e.g., a protease) and wherein at least one of the oligomer blocks of the linked oligomer is an abiotic oligomer block and the enzyme-mediated ligation occurs ex vivo. It will be appreciated that a linked abiotic oligomer of the invention may comprise identical or different telechelic oligomers, both with respect to the primary sequence of monomers in a telechelic oligomer and with respect to the type of telechelic oligomer (e.g., telechelic oligomer comprising amino acids and telechelic oligomer comprising N-substituted glycines).

The present invention does not encompass abiotic linked oligomers generated using recombinant means, such as ribosomal-mediated synthesis in cells or cell free systems or monomer by monomer synthesis. The present method is directed to ligation of telechelic oligomers to generate abiotic linked oligomers.

A novel abiotic linked oligomer of the present invention comprises greater than 65 linked monomers, which form the backbone of the sequence specific heteropolymer. It is believed that abiotic linked sequence specific oligomers greater than 65 linked monomers have not been generated previously. Indeed, the longest abiotic (peptoid) sequence specific heteropolymer reported to date comprised 60 monomers (Lee et al. J Am Chem Soc 2005, 127, 10999-11009). In contrast, the present invention has generated sequence specific heteropolymer products of >150 monomer units in length.

As used herein, the term sequence specific heteropolymer or oligomer refers to copolymers composed of two or more monomer types possessing distinct physico-chemical properties where the arrangement of monomer units is predetermined.

As used herein, the term “concatenate” means to covalently link two distinct block units together, end to end. As used herein, the term concatemer refers to the product of a concatenation reaction; or polymer segments composed of block sequences repeated end to end.

The present invention also encompasses abiotic linked oligomers generated using a method of the present invention.

As used herein, the term “enzyme” is used to refer to a protein that accelerates or catalyzes a reaction. Exemplary enzymes of the invention include, without limitation: oxidoreductases, peroxidases, laccases, transferases, glycotransferases, phosphorylases, glycosyl transferases, acyltransferases, hydrolases, ligases, proteases, lipases, esterases, glycosidases, transaminases and engineered or mutated versions of these natural enzymes.

As used herein, an enzyme recognition element refers to the chemical moiety or moieties on an enzyme substrate that are specifically recognized by the enzyme and which target the substrate for enzyme activity.

As used herein, a protease enzyme refers to any enzyme capable of catalyzing hydrolytic cleavage of a protein(s) into smaller peptide pieces and amino acids by a process known as proteolysis. Exemplary proteases of the invention include clostripain, trypsin, chymotrypsin, V8 protease, subtilisin, subtiligase, collagenase, thermolysin, papain, SGBP, and dipeptidyl peptidase.

As used herein, a protease recognition element refers to the chemical moiety or moieties on a protease substrate that are specifically recognized by the protease and which target the substrate for cleavage by the protease. With respect to the present invention, a protease recognition element refers to chemical groups within one to three monomer units flanking the bond that is generated by the ligation event (corresponding to the scissile bond of the hydrolytic reaction).

As used herein, the terms ligating or linking refer to the formation of a covalent bond between the two or more oligomer units. In particular, for peptoids and peptoid-peptide hybrids, this refers to the formation of an amide bond.

As used herein, “contacting the first and second synthetic oligomers with an enzyme (e.g., a protease) under mild conditions effective to link the first and second synthetic oligomers” refers to conditions approximating or identical to natural biosynthetic environments. For example, aqueous and aqueous/organic mixture solvent environments, buffers (pH 2-14), salts, temperature −20° C. to 120° C. Essentially, the term refers to any or all conditions required for efficient enzyme activity or function or to the general conditions as dictated by requirements for enzyme activity. Minimally, such contacting is performed in the presence of telechelic oligomers, crude or purified enzyme (from either natural or recombinant sources), and a compatible buffer, which comprises an aqueous or aqueous/organic mixture solvent and may include other components, reagents, and cofactors which will allow for efficient enzyme activity.

A “substrate” or “solid support” is a conventional solid support material used in peptide synthesis. Non-limiting examples of such substrates or supports include a variety of solid supports, including Rink Amide resin, Wang resin, Chlorotrityl resin, SPOCC resin, Tentagel resin. Connectors to the solid supports such as those which are photocleavable, DKP-forming linkers (DKP is diketopiperazine; see, e.g., WO90 09395 incorporated herein by reference), TFA cleavable, HF cleavable, fluoride ion cleavable, reductively cleavable and base-labile linkers are also encompassed herein. Planar surface supports, such as paper- or cellulose-based supports, and metal surfaces (such as titanium or gold) are also envisioned. A solid support may also comprise a plurality of solid support particles, such as beads, which can be split into portions or “subamounts” for separate reactions and recombined as desired

As used herein, the terms “immobilized on solid phase” or “solid support-bound” refer to molecules that are attached to a solid phase or solid support. Such attachments may be reversible in nature. A skilled practitioner is familiar with a variety of reversible attachment modes and various protocols to effect release of immobilized molecules from solid supports to which they are attached.

The present invention may be performed either in solution phase or on solid phase. Moreover, sequential ligations may be alternate between solution phase ligation steps and solid phase ligation steps, depending on the desired product (i.e., abiotic linked oligomer). Moreover, sequential reactions may alternate between ligation steps and deprotection steps that expose reactive end-groups of the telechelic oligomer blocks.

The invention also includes a composition for diagnosis or therapy comprising an effective amount of an abiotic linked oligomer of the invention and a physiologically acceptable excipient or carrier. Physiologically acceptable and pharmaceutically acceptable excipients and carriers for use with linked oligomers are well known to those of skill in the art.

By “physiologically or pharmaceutically acceptable carrier” as used herein is meant any substantially non-toxic carrier for administration in which the abiotic linked oligomers of the invention are stable and bioavailable when used. The abiotic linked oligomers can, for example, be dissolved in a liquid, dispersed or emulsified in a medium in a conventional manner to form a liquid preparation or is mixed with a semi-solid (gel) or solid carrier to form a paste, ointment, cream, lotion or the like.

Suitable carriers include water, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, or gum arabic, synthetic polymers, such as discussed below, alcohols, polyols, water and the like. Preferably, because of its non-toxic properties, the carrier is a water miscible carrier composition. Water miscible carrier compositions can include those made with one or more ingredients set forth above but can also include sustained or delayed release carriers, including water containing, water dispersable or water soluble compositions, such as liposomes, microsponges, microspheres or microcapsules, aqueous base ointments, water-in-oil or oil-in-water emulsions or gels.

In one embodiment of the invention, the carrier comprises a sustained release or delayed release carrier. The carrier is any material capable of sustained or delayed release of the abiotic linked oligomer to provide a more efficient administration resulting in one or more of less frequent and/or decreased dosage of the abiotic linked oligomer, ease of handling, and extended or delayed effects. The carrier is capable of releasing the abiotic linked oligomer when exposed to the environment of the area for diagnosis or treatment or by diffusing or by release dependent on the degree of loading of the abiotic linked oligomer to the carrier in order to achieve linked oligomer release. As indicated above, non-limiting examples of such carriers include liposomes, microsponges, microspheres, or microcapsules of natural and synthetic polymers and the like. Examples of suitable carriers for sustained or delayed release in a moist environment include gelatin, gum arabic, xanthane polymers; by degree of loading include lignin polymers and the like; by oily, fatty or waxy environment include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like.

Preferably, the sustained or delayed release carrier is a liposome, microsponge, microphere or gel.

The compositions of the invention are administered by any suitable means, including injection, transdermal, intraocular, transmucosal, bucal, intrapulmonary, and oral. While not required, it is desirable that parenteral compositions maintain the abiotic linked oligomer at the desired location for about 24 to 48 hours; thus, sustained release formulations can be used, including injectable and implantable formulations.

If desired, one or more additional ingredients can be combined in the carrier: such as a moisturizer, vitamins, emulsifier, dispersing agent, wetting agent, odor-modifying agent, gelling agents, stabilizer, propellant, antimicrobial agents, sunscreen, and the like. Those of skill in the art of diagnostic pharmaceutical formulations can readily select the appropriate specific additional ingredients and amounts thereof. Suitable non-limiting examples of additional ingredients include stearyl alcohol, isopropyl myristate, sorbitan monooleate, polyoxyethylene stearate, propylene glycol, water, alkali or alkaline earth lauryl sulfate, methylparaben, octyl dimethyl-p-amino benzoic acid (Padimate O), uric acid, reticulan, polymucosaccharides, hyaluronic acids, aloe vera, lecithin, polyoxyethylene sorbitan monooleate, tocopherol (Vitamin E) or the like.

More particularly, the carrier is a pH balanced buffered aqueous solution for injection. The particular carrier used, however, will vary with the mode of administration.

The compositions for administration usually contain from about 0.0001% to about 90% by weight of the abiotic linked oligomer compared to the total weight of the composition, more particularly from about 0.5% to about 20% by weight of the abiotic linked oligomer compared to the total composition, and even more particularly from about 2% to about 20% by weight of the abiotic linked oligomer compared to the total composition.

The effective amount of the abiotic linked oligomer used for therapy or diagnosis will vary depending on one or more factors such as the specific abiotic linked oligomer used, the age and weight of the patient, the type of formulation and carrier ingredients, frequency of use, the type of therapy or diagnosis preformed and the like. It is a matter of routine for a skilled practitioner to determine the precise amounts to use, taking into consideration these factors and the present specification.

Unless defined otherwise, all technical and scientific terms used herein have the same meaning as commonly understood by one of ordinary skill in the art to which this invention belongs. Although methods and materials similar or equivalent to those described herein can be used in the practice or testing of the invention, preferred methods and materials are described below. The materials, methods, and examples are illustrative only and not intended to be limiting. Other features and advantages of the invention will be apparent from the detailed description, examples, and the claims.

Details Regarding Exemplary Proteases

Protease: Clostripain
Recognition group: 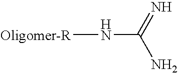
Reaction conditions: 0.0001-1 mM enzyme, 1-10 mM-1 M buffer (eg. HEPES, Tris-HCl), pH 6-10, 0-50% organic solvent (e.g., dimethylformamide, dimethylsulfoxide, acetonitrile), 1-100 mM CaCl₂, 1-100 mM NaCl, 1-100 mM oligomer substrates.
Protease: Trypsin
Recognition group: 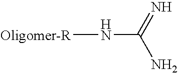
Reaction conditions: 0.0001-1 mM enzyme, 1-10 mM-1 M buffer (e.g., HEPES, Tris-HCl), pH 6-10, 0-50% organic solvent (e.g., dimethylformamide, dimethylsulfoxide, acetonitrile), 1-100 mM CaCl₂, 1-100 mM oligomer substrates.
Protease: Endoproteinase Glu-C, V8 protease
Recognition group: 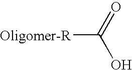
Reaction conditions: 0.0001-1 mM enzyme, 1-10 mM-1 M buffer (e.g., HEPES, Tris-HCl), pH 6-10, 0-50% organic solvent (e.g., dimethylformamide, dimethylsulfoxide, acetonitrile), 1-100 mM oligomer substrates.
Protease: Chymotrypsin
Recognition group: 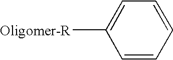
Reaction conditions: Reaction conditions: 0.0001-1 mM enzyme, 1-10 mM-1 M buffer (e.g., HEPES, Tris-HCl), pH 6-10, 0-50% organic solvent (e.g., dimethylformamide, dimethylsulfoxide, acetonitrile), 1-100 mM CaCl₂, 1-100 mM NaCl, 1-100 mM oligomer substrates
   Exemplary Concatemers of Synthetic Sequence-Specific Oligomers

The extracellular matrix provides a structural framework essential for the functional properties of tissues. In each tissue, the three-dimensional organization of the extracellular matrix molecules—elastin, collagens, proteoglycans and structural glycoproteins—synthesized during development and growth is optimal for these functions. Collagen and elastin are comprised of a plurality of repeated domains and are, therefore, excellent candidates for synthetic approaches such as those described in the present invention.

Collagen

Collagen has a characteristic primary, secondary and tertiary structure. Native collagen has a primary structure of repeating trimeric amino acid sequences. The amino acid glycine (Gly) occurs at every third position of a peptide trimer within helical regions, which amount to about 95% of the molecule. Imino residues (I), either proline (Pro) and hydroxyproline (Hyp), occur in 56% of the trimers, 20% as Gly-X-I; 27% as Gly-I-Y; and 9% as Gly-I-I. Pro usually occurs in the second position in the repeating trimer; while Hyp usually occurs in the last position. (Bhatnagar, R. and R. Rapaka (1976) Chap. 10 in Biochemistry of Collagen R. Ramachandren, ed. Plenum Press, New York. pp. 481-482).

Tripeptide sequences (Gly-X-Y) wherein X and Y are amino acid residues other than proline (Pro) or hydroxyproline (Hyp) make up the remaining 44% of the collagen amino acid trimers. Glutamic acid, leucine, and phenylalanine occur most frequently in the X position and threonine, glutamine, methionine, arginine and lysine occur most frequently at the Y position. With the exception of alanine and serine, the X position amino acids have bulky side chains.

Collagen is unusual polypeptide with respect to the secondary and tertiary structure of protein. Most polypeptides comprise sequences of amino acids in peptide linkage which are arrayed either in an α-helix, a right-handed spiral, or alternatively, in a pleated sheet β-conformation. The amino acids of neighboring polypeptide strands are held in place by intramolecular hydrogen bonds in either of these configurations. In contrast, collagen includes three polypeptide chains comprising repeating amino acid trimers. These chains are arrayed in three extended left handed spirals of about three residues per turn, the polyproline II-like chains (Rich et al. (1955) Nature 176:915). The polyproline II-like chains of collagen are arranged in a parallel direction and intertwined to adopt a supercoiled, or coiled coil, right-handed triple helix conformation (Bella et al. (1994) Science 266:75-81) that is characteristic of collagen. The chains that make up the collagen triple helix can be homotrimeric, comprising identical repeating amino acid trimers, or they can be heterotrimeric, comprising chains of different amino acid trimers.

The association of polyproline II-like chains into a triple helix occurs spontaneously; however, the rate of helix formation may be slow due to repulsive “like” charges at the amino and carboxyl ends of the polypeptide sequences that oppose an association of the chains. This “end effect” becomes less important as chain length increases. The rate of helix formation can also be slow because the amino acids in each chain be in “register” properly with each other, and to achieve this they must adjust position appropriately, one along the length of the other. Collagen chains have been found to require a one residue shift between corresponding amino acids in each chain in order to register properly and form trans amides for all peptide bonds.

Synthetic collagens are particularly interesting because they provide materials for collagen-like biomaterials having diverse clinical applications, including use in drug delivery devices, ocular devices, and wound healing materials. Because the proline and hydroxyproline (a post-translationally modified proline residue) residues are abundant in natural collagen sequences, many sequential polymers composed of the trimeric amino acid sequences Gly-Pro-Xaa and Gly-Xaa-Pro (where Xaa is any natural amino acid residue) have been prepared to mimic the collagen structures. Segal and Traub [(1969) J. Mol. Biol. 43:487-496] disclose poly(L-alanyl-L-prolyl-glycine); Segal [(1969) J. Mol. Biol. 43:497-517] discloses collagen-like polyhexapeptides (Gly-Ala-Pro-Gly-Pro-Pro)ₙ, (Gly-Pro-Ala-Gly-Pro-Pro)ₙ, Gly-Ala-Pro-Gly-Pro-Ala)ₙ, and (Gly-Ala-Ala-Gly-Pro-Pro)ₙ; Sakakibara [(1973) Biochim. Biophys. Acta 303:198-202] discloses (Pro-Hyp-Gly)ₙ; Scatturin [(1975) Intl. J. Peptide Protein Res. 7:425-435] discloses (Pro-Leu-Gly)ₙ and (Leu-Pro-Gly)ₙ; Bansal [(1978) Peptide Protein Res. 11:73-81] discloses (Gly-Pro-Leu)ₙ and (Gly-Leu-Pro)ₙ; and Miller [(1980) Macromolecules 13:910-913] discloses poly(glycylprolylalanyl). Ananthanarayanan et al. [(1976) in Chap. 15, Biopolymers, pp. 707-716 (J. Wiley & Sons)] disclose polymers wherein the triplet contains the isomeric N-methyl glycine sarcosine as (Gly-Pro-Sar)ₙ and (Gly-Sar-Pro)ₙ. The above-cited publications are incorporated herein by reference.

Mimicry of natural collagen structures has been directed to enhancing their biostability by inserting unnatural residues into the peptide sequences. To enhance the biostability of collagen-like structures, many unnatural proline analogs and other unnatural imino acid residues, for example, have been used to replace the frequently occurring proline residue in the peptide sequences. Incorporation of such residues, such as the lower homologue of proline, azetidine-2-carboxylic acid (Aze), however, can destabilize the triple helical structure of collagen or prevent its formation (Zagari, A. et al. (1994) Biopolymers, 34:51-60).

Elastin

Elastin, a highly cross-linked complex polypeptide, is a key structural component of the extracellular matrix. Moreover, it is a critical structural and regulatory matrix protein in elastic and muscular arteries, wherein it plays an important and dominant role by conferring elasticity to the vessel wall. Elastin also regulates vascular smooth muscle cell activity and phenotype. Tropoelastin is the soluble precursor of elastin. In addition to providing elastic recoil to various tissues such as the aorta and lung, elastin, tropoelastin and elastin degradation products also influence cell function and promote cellular responses. Elastin is also present in lesser amounts in skin, tendons, and loose connective tissue. In normal mammalian skin, specifically human skin, elastic tissue proteins represent a relatively small fraction of the total dermal proteins, but play a very important role in maintaining the tone, structure, and turgor of the skin.

Elastin fibers are capable of stretching to several times their length and then rapidly returning to their original size upon release of tension. These structural/functional properties, in turn, contribute to the physiological elasticity of tissue comprising elastin. It has been found, for instance, that a loss of elasticity in the skin is associated with a decrease in the tone and turgor of the skin, which is thought to be associated with degradation of elastin and collagen. To address this degradative process, utilization of elastin as a cosmetic agent has been attempted. The dense cross-linked structure of elastin, however, renders solubilization difficult. In fact, elastin is only slightly absorbed by the skin and does not penetrate the skin sufficiently to produce substantial benefits.

The elastin-like polypeptides are a well-defined family of polymers that comprise canonical repeat units. Tropoelastin, the soluble precursor of elastin, is a complex polymeric protein composed primarily of repeating segments of Val-Pro-Gly-Gly, Val-Pro-Gly-Val-Gly, and Ala-Pro-Gly-Val-Gly-Val. It has rubber-like extensible properties. The canonical repeat unit of bovine elastin is Val-Pro-Gly-Val-Gly.

Other Exemplary Concatemers of Synthetic Sequence-Specific Oligomers

In addition to elastin, collagen, and other extracellular matrix biopolymers, numerous repeat peptide/protein motifs are present throughout natural systems and organisms (Main et al. 2005, 15, 464-471). Similar to elastin and collagen, such repeat sequences serve as structural scaffolds having unique and interesting materials properties. These repeat motifs also provide functionality at the molecular level, mediating protein/protein interactions. The following is a non-limiting list of such peptide sequence motifs for which the present invention may be applied in order to generate of long chain macromolecules: abductin family, ankyrin repeat family, antifreeze protein family, armadillo repeat family, circumsporozoite protein family, silk proteins, gliadin, gluten, HEAT repeat, hexapeptide repeat family, leucine rich repeat, peptapeptide repeat family, tetratricopeptide repeat family, resilin, WD40 repeat family.

The abiotic linked oligomers of the invention (e.g., abiotic collagen or abiotic elastin) may be employed for external application and may be used in the form of conventional cosmetic formulations. The abiotic linked oligomers of the invention may also be used in formulations for sub-cutaneous injection.

Although methods and materials similar or equivalent to those described herein can be used in the practice or testing of the invention, preferred methods and materials are described below. The materials, methods, and examples are illustrative only and not intended to be limiting. Other features and advantages of the invention will be apparent from the detailed description and the claims.

### EXAMPLE I

N-substituted glycine oligomers, or peptoids, are an important class of peptidomimetics that demonstrate a propensity to form stable secondary structures (Kirshenbaum et al. Proc. Natl. Acad. Sci. 1998, 95, 4303-4308; Wu et al. J. Am. Chem. Soc. 2003, 125, 13525-13530; Burkoth et al. J. Am. Chem. Soc. 2003, 125, 8841-8845). These “foldamers” (Gellman. Acc. Chem. Res. 1998, 31, 173-180; Hill et al. Chem. Rev. 2001, 101, 3893-4012) display some rudimentary biological activities (Patch et al. Curr. Opin. Chem. Biol. 2002, 6, 872-877; Patch et al. J. Am. Chem. Soc. 2003, 125, 12092-12093; Wender et al. Proc. Natl. Acad. Sci. 2000 97 13003-8) along with resistance to proteolysis (Miller et al. Drug Dev. Res. 1995, 35, 20-32). Further development of peptoids with more sophisticated structure-function relationships, will demand the autonomous folding of tertiary structures exhibiting the complexity of biopolymer architecture (Burkoth et al. J. Am. Chem. Soc. 2003, 125, 8841-8845). Such efforts have been hampered by the difficulties inherent in synthesizing sequence-specific heteropolymers of the requisite chain lengths.

Segment condensation or ligation is an approach that can potentially surmount this hurdle (Horn et al. Helv. Chim. Acta, 2002, 85, 1812-1826; Jang et al. Org. Lett. 2005, 7, 1951-1954; Cheng et al. J. Am. Chem. Soc. 2002, 124, 11564-11565). A number of studies have demonstrated that proteases can be coerced to run in reverse, catalyzing the formation of peptide bonds. Investigations devoted to the reengineering of proteases (Chang et al. Proc. Natl. Acad. Sci. 1994, 91, 12544-12548; Abrahmsen et al. Biochemistry 1991, 30, 4151-4159; Joe et al. Biochemistry 2004, 43, 7672-7677), the design of novel substrates (Sekizaki et al. Tetrahedron Lett. 1997, 38, 1777-1780; Bordusa et al. Angew. Chem. Int. Ed. 1997, 36, 2473-2475; Wehofsky et al. Am. Chem. Soc. 2003, 125, 6126-6133), and the optimization of the reaction medium (Klibanov. Nature. 2001. 409, 241-246) to promote ligation have been reported. The major challenge in utilizing proteases for peptide synthesis is the sequence limitation imposed by the protease—the product itself can be a target for proteolysis. By exploiting the inherent stability of peptoids to proteolytic degradation, protease catalyzed ligation of peptoid segments (oligomer blocks) becomes an attractive approach. Herein, the present inventors demonstrate that protease mediated ligation can be used to generate biomimetic macromolecules through the condensation of peptoid fragments.

The present protocols utilized a protease with its corresponding substrate mimetic (Günther et al. Chem. Eur. J. 2000, 6, 463-467; Günther et al. Org. Chem. 2000, 65, 1672-1679), a chemical moiety resembling the protease's natural substrate. Clostripain, from Clostridium histolyticum, is a cysteine protease which catalyzes the hydrolysis of the amide bond following an arginine residue (Mitchell et al. J. Biol. Chem. 1968, 243, 4683-4692). This enzyme was selected for its broad amino acid sequence tolerance, particularly in the positions adjacent to the scissile bond (Ullmann et al. FEBS 1994, 223, 865-872). p-Guanidinophenyl esters have been reported as substrate mimetics for trypsin, and more recently for clostripain (Sekizaki et al. Tetrahedron Lett. 1997, 38, 1777-1780; Bordusa et al. Angew. Chem. Int. Ed. 1997, 36, 2473-2475; Wehofsky et al. Am. Chem. Soc. 2003, 125, 6126-6133). In the present ligation approach, the p-guanidinophenyl ester serves as a recognition element for the protease, and also acts as an efficient leaving group upon nucleophilic attack by the acyl acceptor peptoid. The catalyzed formation of the amide bond is conducted in competition with the hydrolysis of the p-guanidinophenyl ester.

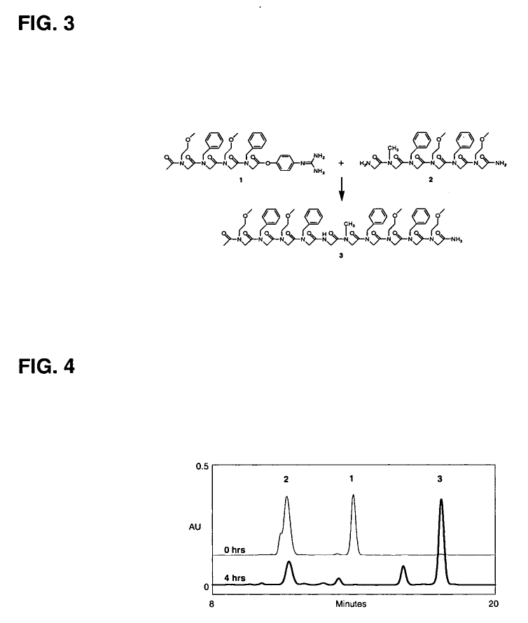

To determine the compatibility of the substrate mimetic approach with peptoid fragments, two model oligomers were evaluated for ligation (FIG. 3; Scheme 1). A peptoid tetramer with a C-terminal acid was synthesized on chlorotrityl resin. The peptoid was cleaved from resin and then modified at the C-terminus with p-guanidinophenol to form the acyl donor 1. The acyl acceptor 2 was prepared using a combination of standard peptoid ‘submonomer’ chemistry (Zuckermann et al. J. Am. Chem. Soc. 1992, 114, 10646-10647) and Fmoc chemistry on Rink amide resin. An unsubstituted glycine residue was positioned at the N-terminus, followed by an N-methyl-glycine (sarcosine) at the subsequent residue. The ligation of 1 and 2 progressed efficiently in the presence of clostripain, as monitored by analytical HPLC (FIG. 4). Complete conversion of the acyl donor was observed within four hours, yielding primarily decamer 3 along with some hydrolysis product. The identity of 3 was confirmed by electrospray mass spectrometry ([M+H⁺], theoretical m/z: 1236.63; calc. m/z: 1237.0). No ligation was observed in the absence of enzyme.

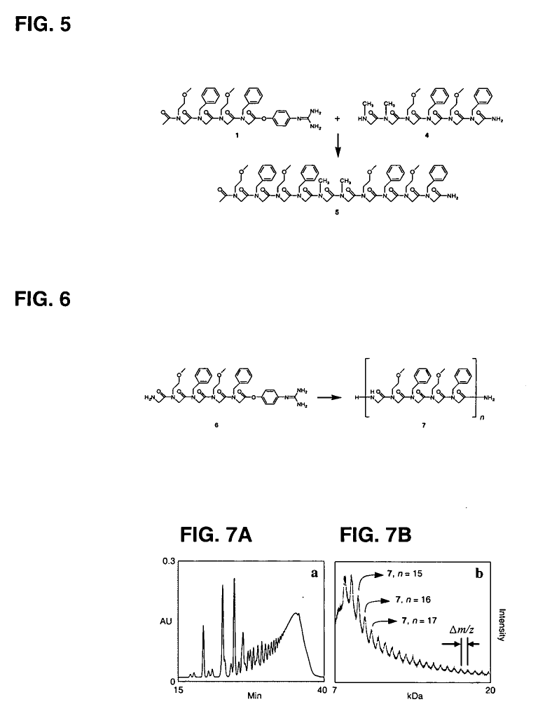

The sequence tolerance for ligation was further examined using peptoid oligomers with N-substituent side chains at all residues (FIG. 5; Scheme 2). The same acyl donor 1 was used, but a different acyl acceptor 4 incorporating two sarcosine monomer units at the N-terminal positions was prepared. Otherwise, conditions were identical as described above. The ligation reaction successfully generated oligomer 5 ([M+H⁺], theoretical m/z: 1250.65; calc. m/z: 1250.9), with a diminished ratio between the ligation and hydrolysis products. Peptide ligation studies suggest that possible steric constraints within or near the active site of clostripain diminish the efficiency of amide bond formation between the donor and acceptor (Ullmann et al. FEBS 1994, 223, 865-872). Nevertheless, the formation of 3 and 5 underscores clostripain's broad substrate tolerance, even towards nonnatural peptidomimetic systems.

The feasibility of protease mediated ligation for the synthesis of long chain sequence-specific polymers was investigated. In reactions ligating two distinct peptoid oligomers, the N-terminus of the acyl donor was acetylated to prevent it from participating in the reaction (FIGS. 3 and 5; Scheme 1 and 2). The present inventors performed ligation reactions with a single peptoid pentamer bearing a free N-terminus and C-terminal p-guanidinophenyl ester. Potentially, oligomer 6 can function as both the acyl donor and acyl acceptor (FIG. 6; Scheme 3). Remarkably, the clostripain catalyzed reaction produced peptoid products exhibiting a range of molecular weights up to and beyond 20 kDa, as confirmed by MALDI-mass spectrometry (FIG. 7B). Mass differences observed between the product molecules 7 correspond to one oligomer repeat unit (average Δm/z=581). The HPLC (FIG. 7A) and MALDI-MS (FIG. 7B) data reveal that the reaction products are the result of numerous iterative ligation events (>30), ultimately forming large concatemers of the starting oligomer.

Protease catalyzed peptide synthesis has been well documented for both L- and D-amino acids as well as for nonnatural residues and small peptide isosteres (Sekizaki et al. Tetrahedron Lett. 1997, 38, 1777-1780; Bordusa et al. Angew. Chem. Int. Ed. 1997, 36, 2473-2475; Wehofsky et al. Am. Chem. Soc. 2003, 125, 6126-6133; Günther et al. Chem. Eur. J. 2000, 6, 463-467; Günther et al. Org. Chem. 2000, 65, 1672-1679; Wehofsky et al. Angew. Chem. Int. Ed. 2003, 42, 677-679). Ribosome-based translation systems have also been utilized for the incorporation of abiotic residues into short peptide sequences (Tan et al. J. Am. Chem. Soc. 2004 126 12752-12753; Frankel et al. Chem. Biol. 2003, 10, 1043-1050). The work presented herein broadens the scope of enzyme based approaches to include the synthesis of nonnatural peptidomimetics. The mild conditions used for these ligation reactions obviates the need for protecting groups on reactive chemical functionalities present on peptidomimetic side chains. This is especially important in the synthesis of long chain polymers, where large numbers of protecting groups can present deprotection and solubility difficulties. Additionally, the approach of the present inventors facilitates the construction of biomimetic polymers of protein biomaterials and other highly repetitive polypeptide sequences. Chimeric polymers containing peptidomimetic fragments with specific chemical functionality may be fused with peptides possessing biological activities for the synthesis of hybrid biocompatible molecules possessing unique properties.

Moreover, improvements in ligation efficiencies may be attained through a variety of alterations: including 1) the use of different enzyme systems (e.g., different proteases, as well as different classes of enzymes, such as lipases, esterases, glycosidases, transaminases); 2) alterations in the reaction medium to comprise different mixtures of organic and aqueous solvents, salt concentration, buffer concentration; and 3) the re-engineering of natural enzymes through recombinant methods to design better catalysts which can better accommodate nonnatural oligomers as substrates.

Ultimately, the present method may facilitate the synthesis of more complex sequence-specific peptoid macromolecules that possess architectural diversity and functionalities comparable to those of proteins. Investigations to elucidate the parameters allowing efficient ligation are currently underway. The parameters include variations in peptoid chain length, sequence, conformation, and substrate mimetic moiety. The synthesis of peptoid concatemers 7 suggests that the ligation approach is generally suitable for assembling peptoid macromolecules. The present inventors believe that the products reported herein are the largest sequence specific peptidomimetics generated to date.

### EXAMPLE II

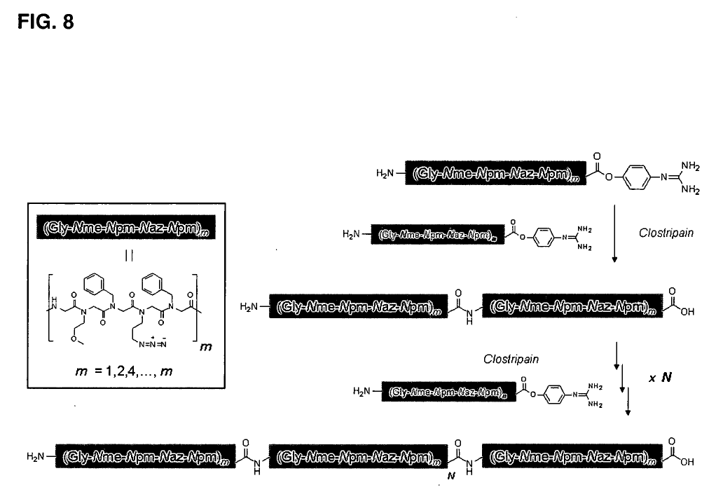

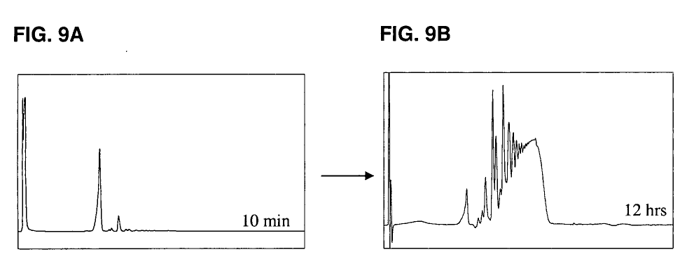

Generation of concatemers of peptoid oligomers containing azide functionality. The present invention, as described above, was used concatenate an abiotic/unnatural oligomer containing an azide functionality (FIG. 8). The abbreviations used in FIG. 8, for example, are standard in the art for peptoid monomers and are as follows: Nme: N-methoxyethyl monomer; Npm: N-phenylmethyl monomer; and Naz: N-azidopropyl monomer. As shown in FIGS. 9A and 9B, the resulting products are polymers of varying chain lengths ranging in size from 2- to n-oligomer units. The largest n-mer produced is greater than 20 kD. The incorporation of a chemically reactive azide group enables these linked oligomer products to be further modified through orthogonal chemistries, which facilitate the attachment of a wide array of molecules. Such molecules range from small low molecular weight molecules to much larger peptides/proteins.

### EXAMPLE III

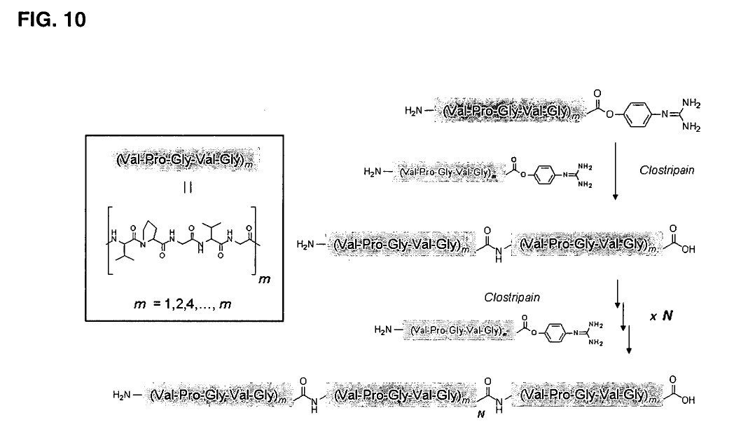

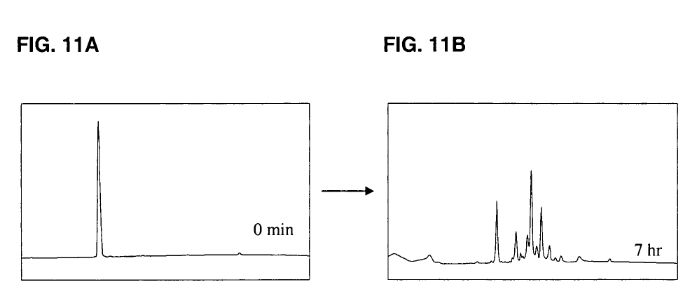

Generation of concatemers of an elastin mimetic peptide. As indicated above, elastin is a naturally occurring class of polypeptides possessing a repeating pentamer motif. The most commonly studied elastin sequence is a repeat of Valine-Proline-Glycine-Valine-Glycine. The present invention was used to concatenate an elastin mimetic peptide comprising Valine-Proline-Glycine-Valine-Glycine. FIG. 10 depicts the concatenation procedure schematically. A graphic representation of HPLC analyses of the progression of the reaction is presented in FIG. 11. The zero (0) minute time point shown in FIG. 11A depicts the elastin peptide (oligomer block comprising amino acids Valine-Proline-Glycine-Valine-Glycine). The plurality of peaks shown in FIG. 11B reveals concatemers of various sizes comprising elastin peptides. The present invention is, therefore, applicable to both ligation and concatenation of peptide sequences.

### EXAMPLE IV

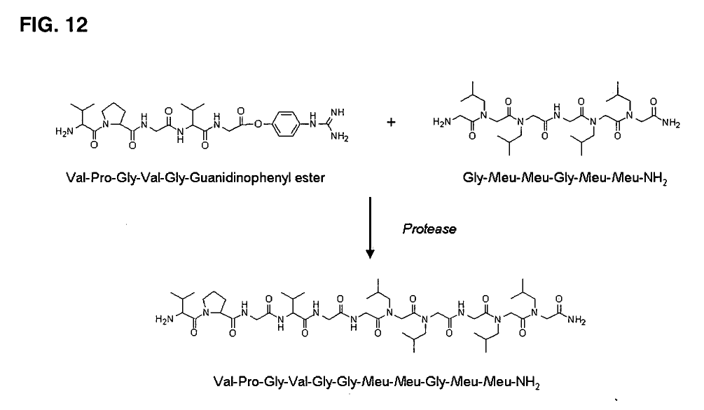

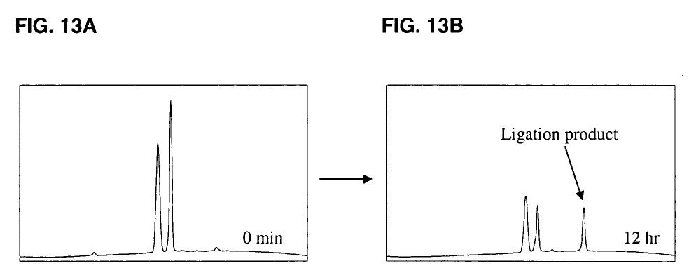

Ligation of peptide oligomers with peptoid oligomers. The present invention was used to join a single peptide and abiotic or unnatural peptoid oligomer. FIG. 12 illustrates the reaction scheme. The reaction was analyzed using HPLC, the results of which are shown in the graphs of FIGS. 13A and 13B. FIG. 13A shows the HPLC profile at the zero (0) time point, wherein the initial starting material, namely the single peptide and abiotic peptoid oligomer are represented by two distinct peaks. After 12 hours reaction time, the presence of a ligation product which is a linked abiotic oligomer comprising a single peptide ligated to a single abiotic peptoid oligomer is apparent. See FIG. 13A. This demonstrates the utility of this invention with respect to synthesizing hybrid polymers containing both natural and unnatural sequences.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed:

1. A method for linking telechelic sequence specific oligomers, the method comprising:
providing a first abiotic telechelic sequence specific oligomer comprising a first enzyme recognition element;
providing a second telechelic sequence specific oligomer;
contacting the first abiotic telechelic sequence specific oligomer and second telechelic sequence specific oligomer with an enzyme capable of recognizing the first enzyme recognition element under mild conditions effective to link the first abiotic telechelic sequence specific oligomer and second telechelic sequence specific oligomer proximate to the enzyme recognition element of the first abiotic telechelic sequence specific oligomer, thereby forming an abiotic linked oligomer comprising the first abiotic telechelic sequence specific oligomer and second telechelic sequence specific oligomer.

2. The method according to claim 1, wherein the first abiotic telechelic sequence specific oligomer is selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas.

3. The method according to claim 1, wherein the second telechelic sequence specific oligomer is an abiotic telechelic sequence specific oligomer.

4. The method according to claim 3, wherein the first and second abiotic telechelic sequence specific oligomers comprise different monomeric sequences.

5. The method according to claim 3, wherein the first and second abiotic telechelic sequence specific oligomers comprise identical monomeric sequences.

6. The method according to claim 1, wherein the enzyme is an oxidoreductase, a peroxidase, a laccase, a transferase, a glycotransferase, a phosphorylase, a glycosyl transferase, an acyltransferase, a hydrolase, a ligase, a protease, a lipase, an esterase, a glycosidase, a transaminase and engineered and mutated versions thereof.

7. The method according to claim 1, wherein the enzyme is a protease and the first enzyme recognition element is a protease recognition element recognized by the protease.

8. The method according to claim 7, wherein the protease is selected from the group consisting of clostripain, trypsin, chymotrypsin, V8 protease, subtilisin, subtiligase, collagenase, thermolysin, papain, SGBP, and dipeptidyl peptidase.

9. The method according to claim 1, wherein the second oligomer comprises a second enzyme recognition element.

10. The method according to claim 9, wherein the first enzyme recognition element and the second enzyme recognition element are identical.

11. The method according to claim 9 further comprising:
repeating said contacting one or more times to generate an abiotic concatenated oligomer comprising at least 3 telechelic sequence specific oligomers.

12. The method according to claim 11, wherein the abiotic concatenated oligomer comprises 3 to 500 telechelic sequence specific oligomers.

13. The method according to claim 3, wherein the first and second abiotic telechelic sequence specific oligomers are selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas.

14. The method according to claim 3, wherein the second abiotic telechelic sequence specific oligomer comprises a second enzyme recognition element.

15. The method according to claim 14, wherein the first enzyme recognition element and the second enzyme recognition element are identical.

16. The method according to claim 14 further comprising:
repeating said contacting one or more times to generate an abiotic concatenated oligomer comprising at least 3 telechelic sequence specific oligomers.

17. The method according to claim 16, wherein the abiotic concatenated oligomer comprises 3 to 500 telechelic sequence specific oligomers.

18. The method according to claim 16, wherein the abiotic concatenated oligomer comprises first and second abiotic telechelic sequence specific oligomers and the first and second abiotic telechelic sequence specific oligomers comprise different monomeric sequences.

19. The method according to claim 16, wherein the abiotic concatenated oligomer comprises first and second abiotic telechelic sequence specific oligomers and the first and second abiotic telechelic sequence specific oligomers comprise identical monomeric sequences.

20. The method according to claim 16, wherein the first and second enzyme recognition elements are protease enzyme recognition elements.

21. The method according to claim 20, wherein the protease that recognizes the first and second enzyme recognition elements is selected from the group consisting of clostripain, trypsin, chymotrypsin, V8 protease, subtilisin, subtiligase, collagenase, thermolysin, papain, SGBP, and dipeptidyl peptidase.

22. An abiotic linked oligomer comprising:
a plurality of first abiotic telechelic sequence specific oligomers; and
a plurality of second telechelic sequence specific oligomers, wherein the first abiotic telechelic sequence specific oligomers and second telechelic sequence specific oligomers are linked and wherein the abiotic linked oligomer comprises more than 65 monomers.

23. The abiotic linked oligomer according to claim 22, wherein the abiotic linked oligomer comprises 2 to 500 first abiotic telechelic sequence specific oligomers and second telechelic sequence specific oligomers.

24. The abiotic linked oligomer according to claim 22, wherein the first abiotic telechelic sequence specific oligomer is selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas.

25. The abiotic linked oligomer according to claim 22, wherein the second telechelic sequence specific oligomers are abiotic telechelic sequence specific oligomers.

26. The abiotic linked oligomer according to claim 25, wherein the first and second abiotic telechelic sequence specific oligomers are selected from the group consisting of N-substituted glycine peptoid oligomers, beta-peptoids, beta-peptides, alternating alpha/beta peptides, gamma-peptides, pyridine oligoamides, quinoline oligoamides, aryl oligoamides, aedemers, peptide nucleic acids, other abiotic oligoamides, sulfonamidopeptides, aminoxy acid oligomers, hydrazone-linked pyrimidines, oligo-ureidophthalimides, triazine-based oligomers, triazole-based oligomers, abiotic polyesters, and oligoureas.

27. The abiotic linked oligomer according to claim 25, wherein the abiotic linked oligomer comprises first and second abiotic telechelic sequence specific oligomers and the first and second abiotic telechelic sequence specific oligomers comprise different monomeric sequences.

28. The abiotic linked oligomer according to claim 25, wherein the abiotic linked oligomer comprises first and second abiotic telechelic sequence specific oligomers and the first and second abiotic telechelic sequence specific oligomers comprise identical monomeric sequences.

29. The abiotic linked oligomer according to claim 25, wherein the abiotic linked oligomer comprises 2 to 500 abiotic telechelic sequence specific oligomers.

30. A method for concatenating telechelic sequence specific oligomers, the method comprising:
providing at least one first telechelic sequence specific oligomer comprising a first enzyme recognition element;
providing at least one second telechelic sequence specific oligomer comprising the first enzyme recognition element;
contacting the at least one first telechelic sequence specific oligomer and the at least one second telechelic sequence specific oligomer with an enzyme capable of recognizing the first enzyme recognition element under mild conditions effective to link first telechelic sequence specific oligomers and second telechelic sequence specific oligomers proximate to the enzyme recognition element of either of the first telechelic sequence specific oligomer or the second telechelic sequence specific oligomer, thereby forming a concatenated oligomer comprising at least 3 first and second telechelic sequence specific oligomers.

31. The method according to claim 30, wherein the first and second telechelic sequence specific oligomers comprise identical monomeric sequences.

32. The method according to claim 30, wherein the first and second telechelic sequence specific oligomers comprise different monomeric sequences.

33. The method according to claim 30, wherein the enzyme is an oxidoreductase, a peroxidase, a laccase, a transferase, a glycotransferase, a phosphorylase, a glycosyl transferase, an acyltransferase, a hydrolase, a ligase, a protease, a lipase, an esterase, a glycosidase, a transaminase and engineered and mutated versions thereof.

34. The method according to claim 30, wherein the enzyme is a protease and the first enzyme recognition element is a protease recognition element recognized by the protease.

35. The method according to claim 34, wherein the protease is selected from the group consisting of clostripain, trypsin, chymotrypsin, V8 protease, subtilisin, subtiligase, collagenase, thermolysin, papain, SGBP, and dipeptidyl peptidase.

36. The method according to claim 31, further comprising contacting the concatenated oligomer comprising at least 3 first and second telechelic sequence specific oligomers with at least one third telechelic sequence specific oligomer comprising the first enzyme recognition element, said contacting the concatenated oligomer and the at least one third telechelic sequence specific oligomer with an enzyme capable of recognizing the first enzyme recognition element under mild conditions effective to link the concatenated oligomer and the at least one third telechelic sequence specific oligomer proximate to the enzyme recognition element of either of the concatenated oligomer or the at least one third telechelic sequence specific oligomer, thereby forming a concatenated oligomer comprising at least 3 first and second telechelic sequence specific oligomers and at least one third telechelic sequence specific oligomer.

37. The method according to claim 32, further comprising contacting the concatenated oligomer comprising at least 3 first and second telechelic sequence specific oligomers with at least one third telechelic sequence specific oligomer comprising the first enzyme recognition element, said contacting the concatenated oligomer and the at least one third telechelic sequence specific oligomer with an enzyme capable of recognizing the first enzyme recognition element under mild conditions effective to link the concatenated oligomer and the at least one third telechelic sequence specific oligomer proximate to the enzyme recognition element of either of the concatenated oligomer or the at least one third telechelic sequence specific oligomer, thereby forming a concatenated oligomer comprising at least 3 first and second telechelic sequence specific oligomers and at least one third telechelic sequence specific oligomer.
